(19) United States
(12) Patent Application Publication
(10) Pub. No.: US 2016/0151023 A1
(43) Pub. Date: Jun. 2, 2016

(54) System and Method for Heart Rate Detection

(71) Applicants: IMEC VZW, Leuven (BE); Katholieke Universiteit Leuven, Leuven (BE)

(72) Inventor: Venkata Rajesh Pamula, Bangalore (IN)

(73) Assignees: IMEC VZW, Leuven (BE); Katholieke Universiteit Leuven, Leuven (BE)

(21) Appl. No.: 14/938,102

(22) Filed: Nov. 11, 2015

(30) Foreign Application Priority Data
Dec. 1, 2014 (EP) 14195689.6
Jan. 21, 2015 (EP) 15151890.9

(51) Int. Cl. A61B 5/00; A61B 5/0245; A61B 5/11; A61B 5/024

(52) U.S. Cl. CPC A61B 5/7278; A61B 5/02405; A61B 5/0245; A61B 5/1102; A61B 5/02416

(57) ABSTRACT

A system for heart rate detection includes a random sampling module configured to provide nonuniform random samples below a Nyquist rate of a biosignal that contains heart rate information; and a heart rate detection module configured to receive a plurality of the nonuniform random samples during a predetermined time window, calculate a power spectral density based on a Lomb-Scargle periodogram of the window samples and calculate a heart rate value based on a frequency corresponding to a highest power peak of the calculated power spectral density. The disclosure also relates to a corresponding method for heart rate detection and a non-transitory computer readable medium.

### CROSS-REFERENCE TO RELATED APPLICATIONS

The present application is a non-provisional patent application claiming priority to European Patent Application No. 14195689.6 filed Dec. 1, 2014, and European Patent Application No. 15151890.9 filed Jan. 21, 2015, the contents of which are hereby incorporated by reference.

### FIELD OF THE DISCLOSURE

The present description relates generally to biosignal acquisition systems and more specifically to systems and methods for heart rate and/or heart rate variability detection with randomly sampled biosignals.

### BACKGROUND

Compressed sensing or compressive sampling (CS) is an emerging signal processing technique that asserts that certain signals can be recovered faithfully from far fewer number of samples or measurements. CS relies on the underlying structure of the signal which is expressed terms of its ‘sparsity’ and the ‘incoherence’ which is related to the sampling scheme (see for example “An Introduction to compressive sampling”, E. J. Candès et al., IEEE Signal Processing Magazine, vol. 25, pp 21-30, March 2008).

Conventional biosignal acquisition systems using, for example, the techniques described in “Compressed Sensing System Considerations for ECG and EMG Wireless Biosensors”, A. M. R. Dixon et al., IEEE Transactions on Biomedical Circuits and Systems, vol. 6, No. 2, April 2012, require, for the detection of a specific biosignal feature, such as for example the heart rate (HR), to first reconstruct an approximation of the original sampled biosignal. This means that complex signal reconstruction algorithms have to be run on the received samples in order to obtain a time domain reconstructed signal and then perform known feature extraction techniques, such as HR or heart rate variability (HRV) detection, on that time domain signal. Such reconstruction process and detection techniques are computationally intensive and hence not suited for low power sensor nodes. Typically, as described in “Artifacts Mitigation in Ambulatory ECG Telemetry”, H. Garudari et al., Proc. IEEE Int. Conf. e-Health Networking Applications and Services, pp. 338-344, 2010, the reconstruction complex processing is offloaded from the sensor and performed at a separated receiver node or base station. With this technique the sensor can work with low power budget while the receiver node, which is assumed to have a better battery budget or no restrictions on power consumption, performs the computationally intensive tasks. Another system describing a HR detector using CS techniques is described in paper “An ultra low power pulse oximeter sensor based on compressed sensing”, P. K. Baheti et al., Body Sensor Networks 2009, pp 144-148, Berkeley, USA 2009.

There is a motivation to improve current state of the art systems and methods in order to reduce HR and/or HRV detection complexity and/or power consumption.

### SUMMARY

The present description provides a new and improved system and method for HR and/or HRV detection from a randomly sampled biosignal that contains HR information, such as for example, an electrocardiogram (ECG), a ballistocardiogram (BCG), or a photoplethysmogram (PPG) signal.

According to an example embodiment, the system and method for HR and/or HRV detection can estimate the HR and/or the HRV from the received measurements of a randomly sampled ECG, BCG, or PPG biosignal without having to reconstruct the original time domain signal.

According to an example embodiment, the system and method for HR and/or HRV detection may reduce HR and/or HRV estimation complexity and/or power consumption compared to state of the art CS techniques.

According to an example embodiment, the system and method for HR and/or HRV detection may reduce the number of samples of the sampled biosignal needed for faithful HR and/or HRV extraction compared to state of the art CS techniques.

According to an example embodiment, the system and method for HR and/or HRV detection may be implemented at the sensor node without significantly increasing the power consumption.

According to an example embodiment, the system is a low power and computationally efficient ECG, BCG or pulseoximetry system capable of estimating HR and/or HRV on the sensor node without having to reconstruct the compressively or randomly sampled data.

According to an example embodiment, there is provided a system for heart rate detection comprising: a random sampling module configured for providing nonuniform random samples below Nyquist rate of a biosignal that contains heart rate information; and a heart rate detection module configured for receiving a plurality of the nonuniform random samples during a predetermined time window and calculating a heart rate value based on the received time window samples; wherein the heart rate detection module is configured to calculate a power spectral density based on a Lomb-Scargle periodogram of the window samples and calculate the heart rate value based on the frequency corresponding to the highest power peak of the calculated power spectral density.

According to an example embodiment, the heart rate detection module may be configured to allocate the received nonuniform random samples into a plurality of windows of samples and calculate the heart rate value for that plurality of window samples.

According to an example embodiment, the heart rate detection module may be configured to search for the highest power peak of the calculated power spectral density of a window of samples within a limited frequency range around an average highest power peak frequency calculated based on a plurality of windows.

According to an example embodiment, the heart rate detection module may be configured to calculate the power spectral density of a window of samples with the use of data stored in a look-up table, the data representing a trigonometric calculation.

According to an example embodiment, the heart rate detection module may be configured to calculate the power spectral density of a window of samples in a frequency band between 0.5 and 5 Hz and with a frequency resolution of 0.08 Hz.

According to an example embodiment, the time interval or window of samples is at least 4 seconds.

According to an example embodiment, heart rate detection module may be further configured to calculate a heart rate variability value as the difference between the heart rate values of two consecutive windows of samples.

There is also provided an electronic device comprising an ECG, BCG or Pulseoximetry system for heart rate detection according to any of the example embodiments herein described.

There is also provided a method for heart rate detection comprising: receiving a plurality of nonuniform random samples below Nyquist rate of a biosignal that contains heart rate information; calculating a power spectral density based on a Lomb-Scargle periodogram of the received plurality of samples; determining a frequency corresponding to the highest power peak of the calculated power spectral density; and calculating a heart rate value based on the determined highest peak power frequency.

According to an example embodiment, the method may further comprise: allocating the plurality of received nonuniform random samples into a plurality of windows of samples and calculating the heart rate value for at least two consecutive windows of samples; and calculating a heart rate variability value as the difference between the heart rate value of the two consecutive windows of samples.

There is also provided a computer program product and a computer readable storage medium according to embodiments of the present description.

### BRIEF DESCRIPTION OF THE FIGURES

Various methods and devices will now be described further, by way of example, with reference to the accompanying drawings, wherein like reference numerals refer to like elements in the various figures.

The above and other aspects of the system and method according to the present description will be shown and explained with reference to the non-restrictive example embodiments described hereinafter.

FIG. 1 shows a general block diagram of an example system for heart rate detection according to the present description.

FIGS. 2A and 2B show, respectively, example graphs of four time domain PPG signal windows and the corresponding four PSD calculations when the time domain PPG signal windows are randomly sampled at an average sampling rate of 12 Hz, corresponding to a Compression Ratio of 10, according to an example embodiment of the present description.

FIGS. 3A and 3B show, respectively, an example graph of a time domain PPG signal window, which is randomly sampled at an average sampling rate of 4 Hz, corresponding to a Compression Ratio of 30 and the corresponding PSD calculation.

FIG. 4 shows an exemplary graph of the HRV value calculated using a method according to an embodiment the present description and a conventional time domain method.

FIG. 5 shows a flow diagram of a method for calculating HR according to an example embodiment of the present description.

FIG. 6 shows a flow diagram of a method for calculating HRV according to an example embodiment of the present description.

### DETAILED DESCRIPTION

In the following, in the description of example embodiments, various features may be grouped together in a single embodiment, figure, or description thereof for the purpose of streamlining the disclosure and aiding in the understanding of one or more of the various inventive aspects. However, embodiments should not be construed as requiring more features those expressly recited in the claims. Furthermore, combinations of features of different embodiments are meant to be within the scope of the disclosure, as would be clearly understood by those skilled in the art. Additionally, in other instances, well-known methods, structures, and techniques have not been shown in detail in order not to obscure the conciseness of the description.

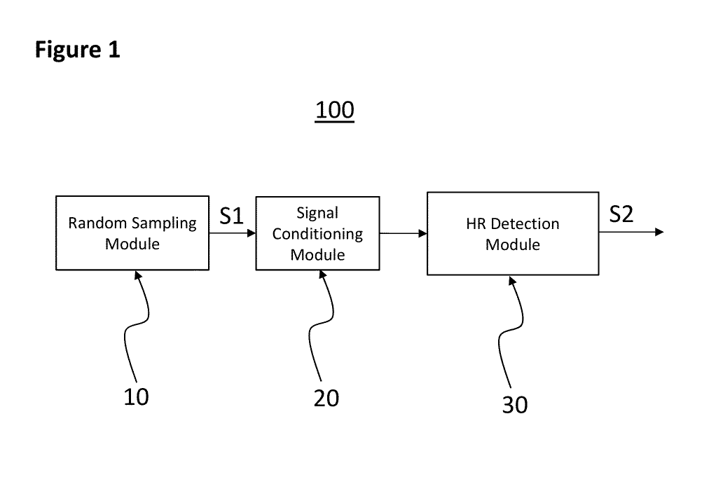

FIG. 1 shows a general block diagram of an example system for heart rate detection 100 comprising a random sampling module 10 providing a randomly sampled biosignal S1, a signal conditioning module 20, and a HR detection module 30 providing information about a subject's HR and/or HRV S2. The random sampling module 10 may include a sensor that generates an analog version of a certain sensed biosignal, e.g. ECG, BCG or PPG and the signal is then sampled according to current state of the art nonuniform random sampling techniques (below Nyquist sampling rate) so that a random-sampled version S1 of the sensed biosignal is provided to the next signal conditioning, transmission and/or processing stages. References for nonuniform random sampling techniques can be found in “Compressive Sensing by Random Convolution”, by J. Romberg, SIAM Journal on Imaging Sciences, vol. 2, no. 4, October 2009; and “Sparsity and Incoherence in Compressive Sampling”, by E. Candes and J. Romberg, Inverse Prob., vol. 23, no. 3, pp. 969-985, 2007. Alternatively, the random sampling module 10 may trigger or activate a sensor according to current state of the art nonuniform random sampling techniques (below Nyquist sampling rate) so that the sensor directly generates a random sampled version S1 of the sensed biosignal. The signal conditioning module 20 may be optional and comprise an analogue to digital converter and/or any other element for conditioning of the randomly sampled signal S1 to a transmission means. The HR detection module 30 is adapted to receive information concerning the random sampled signal S1, e.g. value and time stamp when the signal was sampled, and process the received samples in order to calculate an estimation of the HR and/or HRV S2. According to an embodiment, the HR detection module 30 calculates an estimation of the HR based on the spectral information of the random sampled signal S1. According to an example embodiment, the HR detection module 30 performs least-squares frequency analysis of the random sampled signal S1 in order to calculate an estimation of the HR. Least-squares spectral analysis (LSSA) or Lomb-Scargle periodogram is a method of estimating a frequency spectrum of unequally spaced data as described, for example, in “Least-squares Frequency Analysis of Unequally Spaced Data”, N. R. Lomb, Astrophysics and Space Science 39, 447-462, 1976, in which the power spectral density (PSD) of the samples is calculated using:

$$\begin{matrix}P ( ω ) = \frac{1}{2} \{ \frac{{( \sum x ( {t}_{j} ) cos ω ( {t}_{j} - τ ) )}^{2}}{\sum {cos}^{2} ω ( {t}_{j} - τ )} + \frac{{( \sum x ( {t}_{j} ) sin ω ( {t}_{j} - τ ) )}^{2}}{\sum {sin}^{2} ω ( {t}_{j} - τ )} \} & ( 1 ) \\ tan ( 2 ω τ ) = \frac{\sum sin 2 ω tj}{\sum cos 2 ω tj} & ( 2 )\end{matrix}$$

where x(tⱼ) is the signal at time instant tⱼ (jth sample of the signal) and ω is the frequency at which the PSD is to be estimated in rad/sec.

According to an example embodiment, the HR detection module 30 calculates the PSD of a plurality of samples of the received randomly sampled signal S1 and infers the HR information from the same. According to an example embodiment, an average HR over a certain predetermined time interval or window, e.g., 4 seconds, is calculated by determining the frequency (fpk) corresponding to the highest power peak in the PSD of the samples received during that time period or window and then calculating the HR, in beats per minute (bpm), as:

HR=60×fpk (3)

According to an example embodiment, the HR detection module 30 may calculate the HRV based on the analysis of a plurality of, e.g., at least two, consecutive time windows comprising samples of the randomly sampled signal S1. Each window contains the samples received in a certain time period, e.g., 4 seconds, and the HR detection module 30 calculates the spectral density (PSD) of those samples and infers the HR for each window. Then the HRV is calculated as the difference between the HR on the consecutive windows. The plurality of consecutive time windows may be overlapping or non-overlapping time windows.

According to an example embodiment, the system for heart rate detection 100 may provide an accurate HR/HRV estimation S2 from randomly sampled biosignal data S1 without having to reconstruct an approximation of the original continuous-time biosignal (in the time domain).

According to an example embodiment, the system for heart rate detection 100 may be used for accurate HR/HRV estimation S2 from randomly sampled PPG data S1 thereby leading to a low complexity, low power pulseoximeter system. It will be noted that conventional pulseoximeters may employ uniform sampling and estimate the HR and HRV from those acquired (at Nyquist sampling rate) uniform samples. On the other hand, for pulseoximeters employing compressive sampling techniques, current techniques also rely on a full reconstruction of the original time domain signal in order to perform a subsequent extraction of HR and/or HRV information. Such approaches are computationally intensive and are not suited for implementation on low power sensor nodes with small form factors. Furthermore, in certain applications, a full reconstruction of the originally sensed biosignal is not required provided the vital information, such as HR/HRV can be extracted, as is explained for the embodiments of the present description.

According to an example embodiment, the system for heart rate detection 100 may estimate the HR and/or HRV with a high degree of confidence even at very high compression ratios (e.g. greater than 30) of the randomly sampled biosignal data S1, where the conventional reconstruction methods fail to reconstruct the signal accurately. It will be noted that reconstruction techniques based on compressive sampling require a minimum number of samples or measurements in order to be able to faithfully reconstruct the original time domain biosignal. The theory of CS asserts that M out N samples are sufficient for a K-sparse signal for faithful reconstruction, provided M=O(K log (N/K)<<N, which puts a limit on the achievable compression ratio (CR=N/M) which guarantees faithful reconstruction. Such limitation is overcome with embodiments of the present description. It will be noted that there must be a minimum number of entries in the random sampling matrix in order to be able to estimate the PSD. The boundary on the same depends on the type of biosignal and the required HR/HRV detection accuracy, which can be determined by the person skilled in the art. Additional information can be obtained in “A Prescription for Period Analysis of Unevenly Sampled Time Series”, by J. H. Horne and S. L. Baliunas, The Astrophysical Journal, 302: pp. 757-763, March 1986.

The HR estimated according to example embodiments of the present description provides an accuracy of around ±5 bpm compared to HR estimation based on time domain analysis. HRV calculated according to example embodiments of the present description shows a correlation to HRV estimation based on the time domain signal analysis, with a correlation coefficient greater than 0.95 for a compression ratio of 10 and greater than 0.9 for a compression ratio of 30.

According to an example embodiment, the HR detection module 30 may be adapted to search across the entire frequency band of the calculated PSD in order to find the highest power peak frequency (fpk). According to another example embodiment, the HR detection module 30 may be further adapted to search in a narrower frequency band around an average highest power peak frequency (afpk) calculated based on a plurality of windows. According to an example embodiment, the HR detection module 30 may be adapted to calculate the PSD and find the frequency corresponding to the highest power peak (fpk) of four windows of samples and then calculate an average highest power peak frequency (afpk) based on the previous four highest power peak frequencies. Subsequently, for successive windows of samples, the HR detection module 30 may be adapted to just search for the highest PSD power peak frequency (fpk) within ±1 Hz of the calculated average highest power peak frequency (afpk). According to an example embodiment, the four windows of samples are consecutive and non-overlapping windows of samples. With this adaptive peak/frequency search scheme, the system may reduce the false estimation of the HR/HRV in presence of motion artifacts, which makes the system more robust and accurate.

According to an example embodiment, the HR detection module 30 may be further adapted to calculate the PSD of the window samples based on values stored in a look-up table. According to an example embodiment, the system for heart rate detection 100 has already knowledge by design of the random sampling instances (tⱼ) and terms τ, ω and hence cos ω (τ−tⱼ) and sin ω (τ−tⱼ) may be calculated in advance and stored in a look-up table. The pre-calculation is based on the fact that the system is expected to work over a known range of HR (30 bpm-300 bpm), so the PSD is to be estimated only across a narrow band of frequency ranges from 0.5 Hz to 5 Hz. The frequency resolution is also determined by the desired accuracy in estimating HR, which is typically ±5 bpm (as per ANSI-AAMI standards), thereby requiring a frequency resolution of 0.08 Hz. Henceforth the frequency interval [0.5, 5] can be divided into linear steps with step size being 0.08 Hz. This technique, according to an example embodiment of the present description, may simplify the computation of the PSD since it eliminates or reduces the need for computation of complex trigonometric quantities using CORDIC based methods. This increases the throughput and further reduces complexity and power consumption at the sensor node.

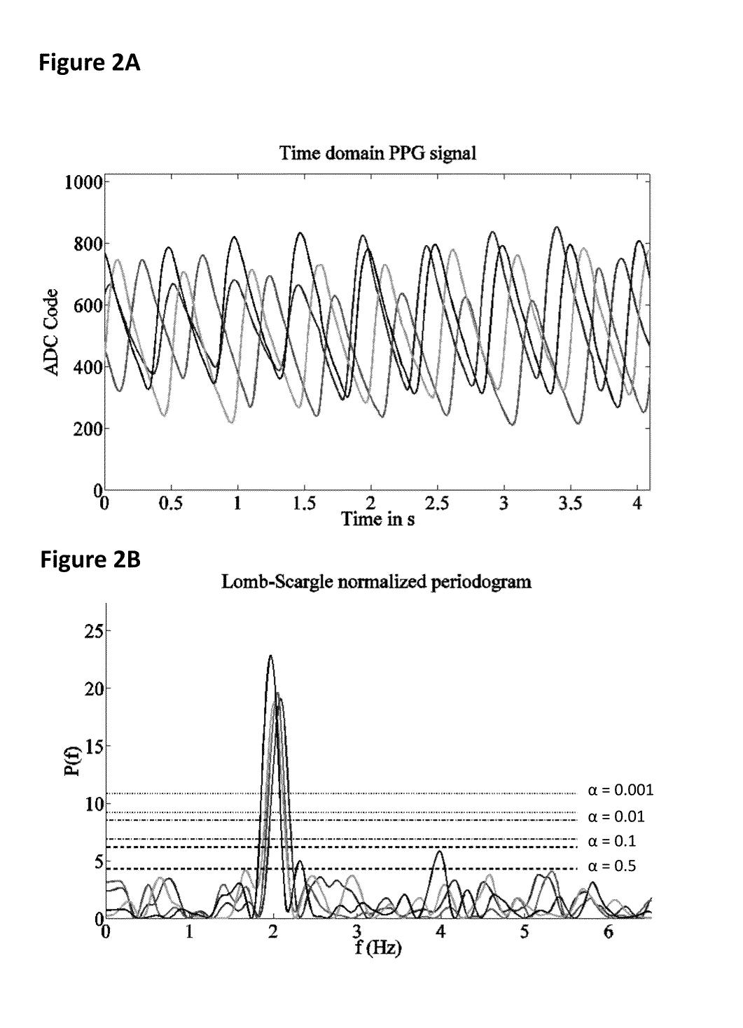

FIG. 2A shows an example graph of four superimposed time domain PPG signal windows of 4 seconds. Each PPG signal window is part of a time domain PPG signal of 16 seconds that has been sliced into four consecutive 4-second signal windows according to an example embodiment of the present description. FIG. 2B shows four superimposed PSD calculations of the four PPG signal windows of FIG. 2A when the time domain signal windows are randomly sampled at an average rate of 12 Hz (corresponding to a CR of 10). According to an example embodiment, the HR estimation, calculated over each period of 4 seconds, is then calculated by determining the frequency corresponding to the highest peak in the PSD (fpk) and then calculating the HR in bpm as HR=60×fpk. It will be noted that the time period for HR/HRV calculations may vary, e.g. calculation every 5 or 10 seconds, and may be set by the designer or user, depending on the desired implementation.

According to an example embodiment, the HR detection module 30 calculates a HR value based on the PSD of the samples received during a time window of at least 4 seconds. This represents at least two cycles of a biosignal with a minimum HR of 30 bpm (the period being 2 seconds). For a better estimation of the HR, more than 2 cycles are desirable. Therefore, longer time windows for HR estimation, e.g. 16 seconds, may be used, but this may lead to larger latency and a lower update rate, i.e. the system can provide HR and/or HRV information only every 16 seconds. HRV may be calculated as the difference between the HR calculated in two consecutive windows of FIG. 2A.

The confidence level in the estimation of the PSD is given by 1−α, where α is the probability of estimation being false. It can be observed in FIG. 2B that the confidence level in estimation of the PSD is above 99% even at a CR of 10.

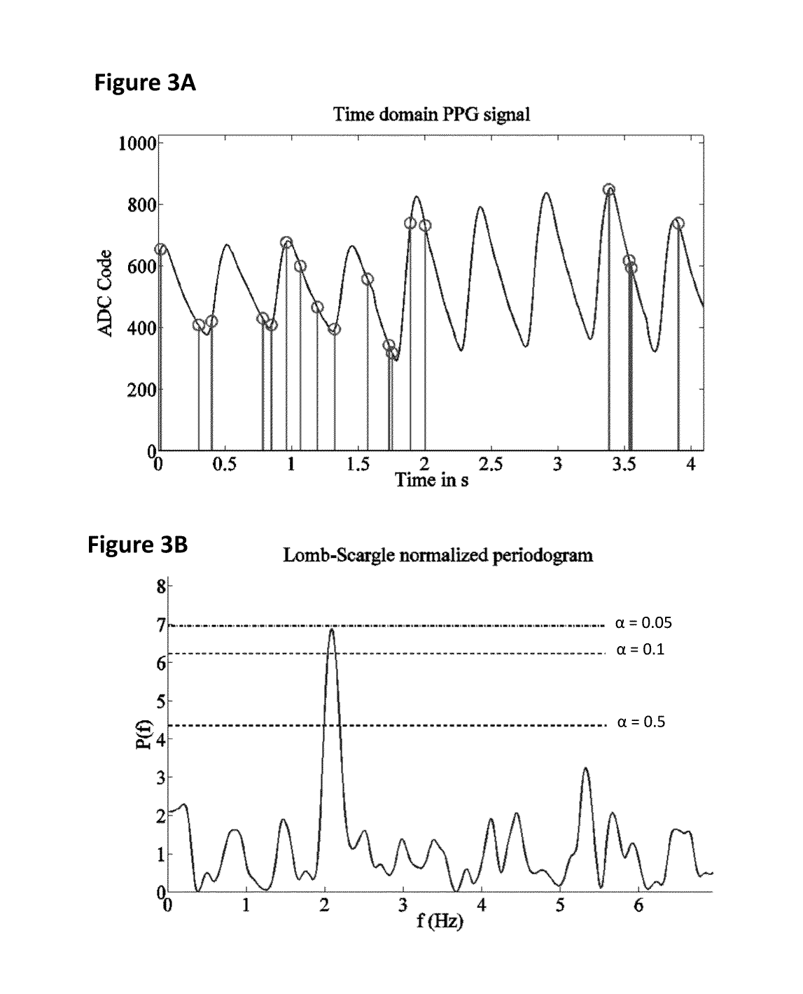

FIG. 3A shows an example graph of a time domain PPG signal window of 4 seconds, which is randomly sampled according to a CR of 30. FIG. 3B shows the PSD calculation of the randomly sampled time domain PPG signal window of FIG. 3A. It can be seen that there is a predominant peak in the PSD corresponding to the average HR. Although the confidence level in the PSD estimation (95%) is lower than the example of FIGS. 2A and 2B for a CR of 10, it may be still sufficient for certain applications. The described example method for estimation of the HR can then be extended to high compression ratios, e.g., 30, where the conventional methods based on time domain signal reconstruction fail to give satisfactory reconstructed signal quality. Furthermore, according to an example embodiment, the system for heart rate detection 100 can be designed adaptively to achieve a certain trade-off between compression ratio and the desired confidence level in estimation of PSD.

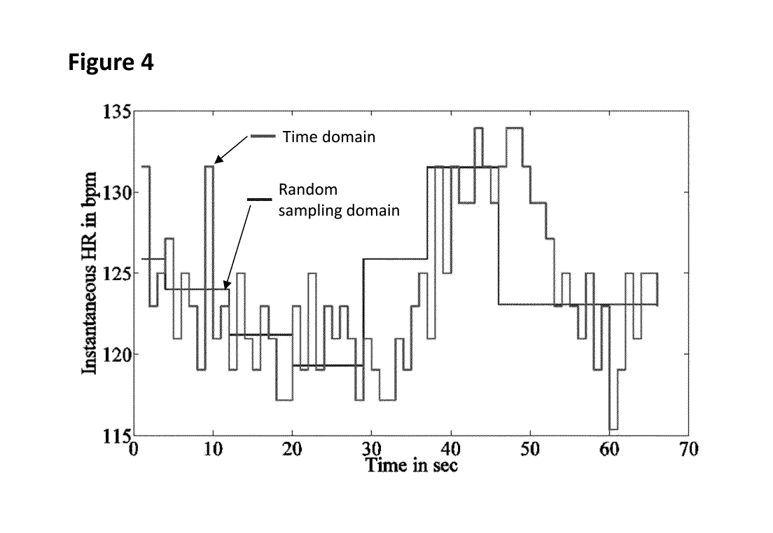

FIG. 4 shows an example graph for comparison of the HRV value as a function of time for an example measured PPG signal, when the HRV is computed using a method according to an embodiment the present description or a conventional time domain method. According to an example embodiment, the original PPG signal is sampled at 125 Hz, while the proposed method of HR calculation is implemented on the original signal after randomly sampling it to obtain a CR of 10, which corresponds to an average sampling rate of 12 Hz. The time window for calculation of the PSD is 4 seconds and the HRV is computed based on non-overlapping windows.

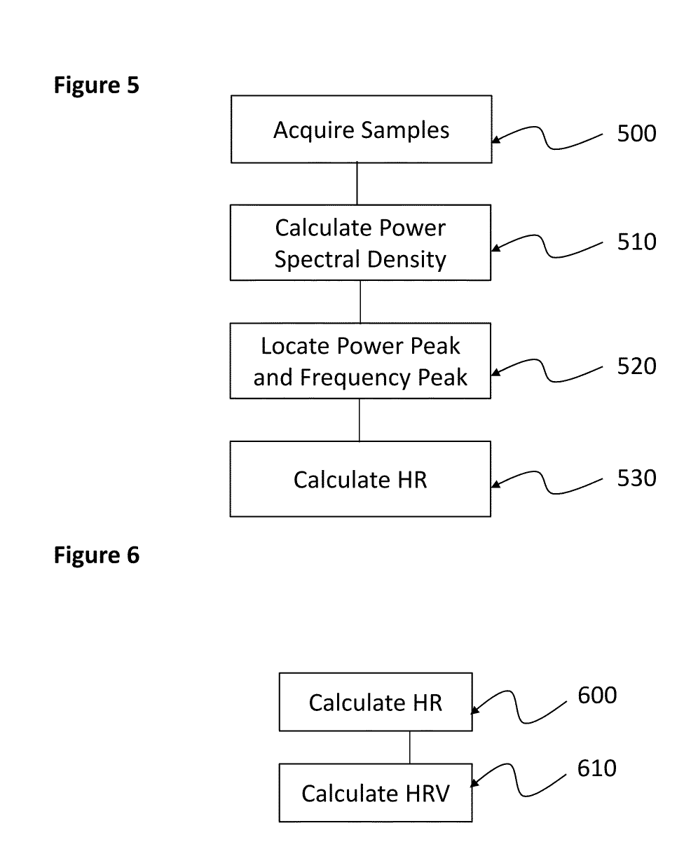

FIG. 5 shows a flow diagram of a method for calculating HR according to an example embodiment of the present description. In block 500 a plurality of random samples of the biosignal S1 are acquired during a time interval or window of T seconds. In block 510 the Lomb-Scargle periodogram is calculated as in equations (1) and (2) (explained with reference to FIG. 1). According to an example embodiment, the Lomb-Scargle periodogram is calculated for a frequency range between 0.5 to 5 Hz and with a resolution of 0.08 Hz. In block 520 a search is done in order to locate the highest power peak of the Lomb-Scargle periodogram and the corresponding frequency (fpk). According to an example embodiment, in the presence of motion artifacts, the search for the highest power peak frequency (fpk) may be limited around ±1 Hz of a previously calculated average highest power peak frequency (afpk) based on a plurality of window samples. In block 530, the HR is calculated based on the highest power peak frequency (fpk) as in equation (3) (explained with reference to FIG. 1). Blocks 500 to 530 may be repeated as long as HR information S2 is to be calculated and provided.

FIG. 6 shows a flow diagram of a method for calculating HRV according to an example embodiment of the present description. In block 600 HR values are calculated according to the example method shown in FIG. 5. Then, in block 610 a HRV value is calculated as the difference between the HR of two consecutive windows of samples. Block 610 may be repeated as long as HRV information S2 is to be calculated and provided.

It will be noted that the HR detection module 30 may be implemented according to hardware and/or software state of the art techniques, comprising for example a microprocessor, microcontroller or digital signal processor that can understand and execute software program instructions. Some programmable hardware logic and memory means may be specifically designed also for executing the method or parts of it according to example embodiments of the present description.

A step or block that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical functions or actions in the method or technique. The program code and/or related data can be stored on any type of computer readable medium such as a storage device including a disk, hard drive, or other storage medium.

The computer readable medium can also include non-transitory computer readable media such as computer-readable media that store data for short periods of time like register memory, processor cache, and random access memory (RAM). The computer readable media can also include non-transitory computer readable media that store program code and/or data for longer periods of time. Thus, the computer readable media may include secondary or persistent long term storage, like read only memory (ROM), optical or magnetic disks, compact-disc read only memory (CD-ROM), for example. The computer readable media can also be any other volatile or non-volatile storage systems. A computer readable medium can be considered a computer readable storage medium, for example, or a tangible storage device.

What is claimed is:

1. A system for heart rate detection comprising:
a random sampling module configured to provide nonuniform random samples below a Nyquist rate of a biosignal that contains heart rate information; and
a heart rate detection module configured to:
receive a plurality of the nonuniform random samples during a predetermined time window;
calculate a power spectral density based on a Lomb-Scargle periodogram of said the received nonuniform random samples; and
calculate a heart rate value based on a frequency corresponding to a highest power peak of the calculated power spectral density.

2. The system for heart rate detection according to claim 1, wherein the heart rate detection module is configured to allocate the received nonuniform random samples into a plurality of windows of samples, and calculate the heart rate value for at least one window of samples based on the received nonuniform random samples allocated to the at least one window of samples.

3. The system for heart rate detection according to claim 1, wherein the heart rate detection module is configured to search for the highest power peak of the calculated power spectral density of a respective window of samples within a limited frequency range around an average highest power peak frequency calculated based on a plurality of windows of samples.

4. The system for heart rate detection according to claim 1, wherein the heart rate detection module is configured to calculate the power spectral density of a window of samples based on data stored in a look-up table, wherein the data represents a trigonometric calculation.

5. The system for heart rate detection according to claim 1, wherein the heart rate detection module is configured to calculate the power spectral density of a window of samples in a frequency band between 0.5 Hz and 5 Hz, and with a frequency resolution less than or equal to 0.08 Hz.

6. The system for heart rate detection according to claim 1, wherein the window of samples comprises a time window of at least 4 seconds.

7. The system for heart rate detection according to claim 1, wherein the heart rate detection module is further configured to calculate a heart rate variability value as the difference between the respective heart rate values of two consecutive windows of samples.

8. The system for heart rate detection according to claim 1, wherein the biosignal that contains heart rate information is an ECG, BCG, or PPG signal.

9. The system for heart rate detection according to claim 1, wherein the system is arranged as at least one of an electronic device or a network of electronic devices.

10. A method for heart rate detection comprising:
receiving a plurality of nonuniform random samples below a Nyquist rate of a biosignal that contains heart rate information;
calculating a power spectral density based on a Lomb-Scargle periodogram of the received plurality of nonuniform random samples;
determining a frequency corresponding to a highest power peak of the calculated power spectral density; and
calculating a heart rate value based on the determined highest peak power frequency.

11. The method for heart rate detection according to claim 10, further comprising:
allocating the received plurality of nonuniform random samples into a plurality of windows of samples and calculating respective heart rate values for at least two consecutive windows of samples; and
calculating a heart rate variability value as a difference between the respective heart rate values of the at least two consecutive windows of samples.

12. A non-transitory computer readable medium having stored therein instructions executable by a computer system to cause the computer system to perform functions comprising:
receiving a plurality of nonuniform random samples below a Nyquist rate of a biosignal that contains heart rate information;
calculating a power spectral density based on a Lomb-Scargle periodogram of the received plurality of nonuniform random samples;
determining a frequency corresponding to a highest power peak of the calculated power spectral density; and
calculating a heart rate value based on the determined highest peak power frequency.

13. The non-transitory computer readable medium according to claim 12, wherein the functions further comprise:
allocating the received plurality of nonuniform random samples into a plurality of windows of samples and calculating respective heart rate values for at least two consecutive windows of samples; and
calculating a heart rate variability value as a difference between the respective heart rate values of the at least two consecutive windows of samples.
